(19) United States
(12) Patent Application Publication
(10) Pub. No.: US 2002/0031532 A1
(43) Pub. Date: Mar. 14, 2002

(54) SHAMPOO COMPOSITION COMPRISING SILICONE EMULSION

(76) Inventor: HIROTAKA UCHIYAMA, KOBE (JP)

Correspondence Address: LINDA M. SIVIK-BOX 325, THE PROCTER & GAMBLE COMPANY, 11511 REED HARTMAN HIGHWAY, SHARON WOODS TECHNICAL CENTER, CINCINNATI, OH, 45241, US

(21) Appl. No.: 09/297,508

(22) PCT Filed: Nov. 4, 1996
(86) PCT No.: PCT/US96/17578

(51) Int. Cl. A61K 7/06; A61K 7/11; A61K 7/075; A61K 7/08; A61K 6/00; A61K 7/00

(52) U.S. Cl. 424/401; 424/70.1; 424/70.11; 424/70.12; 424/70.122; 424/70.13; 424/70.19; 510/119; 510/122; 510/123; 510/129

(57) ABSTRACT

Disclosed are shampoo compositions comprising a silicone emulsion comprising a silicone polymer selected from the group consisting of a polyalkyl siloxane having a molecular weight of at least 20,000, a polyaryl siloxane having a molecular weight of at least 20,000, and amino-substituted siloxane having a molecular weight of at least 5,000, a silicone resin having a molecular weight of at least 5,000, and mixtures thereof, an anionic surfactant, a compatibilizing surfactant, and a cationic surfactant, Wherein the silicone polymer is dispersed as a particle having an average size of not more than 450 nm; a detersive surfactant; a conditioning agent; and water: wherein the composition is substantially free of silicone suspending agents.

### TECHNICAL FIELD

The present invention relates to a shampoo composition comprising a silicone emulsion.

### BACKGROUND

Human hair becomes soiled due to its contact with the surrounding environment and from the sebum secreted by the scalp. The soiling of hair causes it to have a dirty feel and an unattractive appearance. The soiling of the hair necessitates shampooing with frequent regularity.

Shampooing cleans the hair by removing excess soil and sebum. However, shampooing can leave the hair in a wet, tangled, and generally unmanageable state. One the hair dries, it is often left in a dry, rough, lusterless, or frizzy condition due to removal of the hair's natural oils and other natural conditioning and moisturizing components. The hair can further be left with increased levels of static upon drying which can interfere with combing and result in a condition commonly referred to as “fly-away hair”, or contribute to an undesirable phenomena of “split ends”, particularly for long hair.

A variety of approaches have been developed to alleviate these after-shampoo problems. These approaches range from post-shampoo application of hair conditioner such as leave-on and rinse-off products, to hair conditioning shampoos which attempt to both cleanse and condition the hair from a single product. Hair conditioners are typically applied in a separate step following shampooing. The hair conditioners are either rinsed-off or left-on, depending upon the type of product used. Hair conditioners, however, have the disadvantage of requiring a separate and inconvenient treatment step. Conditioning shampoos are highly desirable products because they are convenient for consumers to use by providing cleansing and conditioning benefits to the hair in one step.

In order to provide hair conditioning benefits in a cleaning shampoo base, a wide variety of conditioning actives have been proposed. However, they have not been totally satisfactory.

One problem relates to compatibility between anionic detersive surfactants and the many conventional cationic conditioning agents. Whereas efforts have been made to minimize adverse interaction through the use of alternative surfactants, it remains highly desirable to utilize anionic surfactants to some extent because of its overall superior cleaning properties. On the other hand, some consumers desire mild or non-stimulating shampoo compositions which usually comprise other classes of surfactants in addition to said anionic surfactants. Thus, a shampoo composition which is compatible with a wide variety of detersive surfactants is desired.

Materials which can provide improved overall conditioning benefits while maintaining cleaning performance with the use of anionic detersive surfactants are silicone conditioning agents. However, shampoos comprising silicone conditioning agents have a tendency of providing undesirable feeling to the hair such as leaving the hair feeling coated, heavy, or soiled after the hair is dried. Further, in order to provide a well dispersed, storage stable shampoo composition including silicone conditioning agents, a suspending agent such as acyl derivatives is required. The combination of silicone conditioning agents and its suspending agents often provide a formulation which is relatively viscous and milky in appearance. This is particularly noticeable for syspending agents such as ethylene glycol stearates.

The undesired feeling to the hair as well as the unstability observed for silicone conditioning agents is thought to be due to the particle size of the silicone conditioning agent. This is particularly noticeable when the silicone has a high molecular weight. Although high molecular weight silicone polymers are known to have favorable conditioning benefits such as smoothness and combing ease, they also tend to have a large particle size and are thermodynamically unstable. Mechanical shearing is known to provide smaller particle size of fluids. High molecular weight silicone polymers are too viscous to emulsify down to a desirable particle size. Thus, high molecular weight silicone polymers, without the aid of a suspending agent, could not be formulated at levels that would provide desired conditioning benefits.

Therefore, there remains a desire to provide a shampoo composition comprising high molecular weight silicone polymers which are stable without suspending agents and provide overall improved conditioning benefits.

Japanese Patent Laid-open 7-138,136 discloses a hair cleaning composition comprising a surfactant, and a water-insoluble highly polymerized silicone emulsion obtained by emulsion polymerization and having an average particle size of 0.2-50 microns. European Patent Application 674,898-A discloses a conditioning shampoo composition for hair comprising a stable microemulsion of a high viscosity silicone with a particle size of less than 0.15 microns, in combination with a deposition polymer and a surfactant. U.S. Pat. No. 5,504,149 discloses a method for making a silicone emulsion having high viscosity wherein a mixture of water, cyclic siloxane, optional nonionic surfactant and cationic surfactant is polymerized by using silanolate or organosilanolate as an initiator.

In the present invention, a shampoo composition comprising a silicone emulsion comprising a high molecular weight silicone polymer made via a certain surfactant system have been developed which provide are stable without silicone suspending agents and provide overall improved conditioning benefits by being compatible with a wide range of conditioning agents.

### SUMMARY

The present invention relates to a shampoo composition comprising by weight:

(a) a silicone emulsion comprising:

i) from about 0.01% to about 20% of the entire composition a silicone polymer selected from the group consisting of a polyalkyl siloxane having a molecular weight of at least 20,000, a polyaryl siloxane having a molecular weight of at least 20,000, an amino-substituted siloxane having a molecular weight of at least 5,000, a silicone resin having a molecular weight of at least 5,000, and mixtures thereof;

ii) an anionic surfactant;

iii) a compatibilizing surfactant; and

iv) a cationic surfactant; wherein the silicone polymer is dispersed as a particle having an average size of not more than about 450 nm;

(b) from about 5% to about 50% of a detersive surfactant;

(c) from about 0.1 to about 20% of a conditioning agent; and

(d) water;

wherein the composition is substantially free of acyl derivative silicone suspending agents.

Such compositions satisfy the need for a hair conditioning composition which has overall improved conditioning benefits, and which can be used with a wide range of conditioning agents without acyl derivative silicone suspending agents.

### DETAILED DESCRIPTION

All percentages herein are by weight of the compositions unless otherwise indicated. All ratios are weight ratios unless otherwise indicated. All percentages, ratios, and levels of ingredients referred to herein are based on the actual amount of the ingredient, and do not include solvents, fillers, or other materials with which the ingredient may be combined as commercially available products, unless otherwise indicated.

The invention hereof can comprise, consist of, or consist essentially of the essential elements described herein as well as any of the preferred or optional ingredients also described herein.

All publications, patent applications, and issued patents mentioned herein are hereby incorporated in their entirety by reference.

Silicone Emulsion

The shampoo composition of the present invention comprises a silicone emulsion comprising a silicone polymer, an anionic surfactant; a compatibilizing surfactant, and a cationic surfactant. The silicone emulsion is prepared by emulsion polymerization, wherein an aqueous solution or emulsion of the starting silicone material is mixed with an anionic surfactant, followed by addition of a compatibilizing surfactant, and finally by addition of a cationic surfactant. The starting silicone material is selected so that the resulting silicone polymer in the obtained silicone emulsion has more than a certain molecular weight, and dispersed as a particle having an average size of not more than about 450 nm, more preferably about from 150 nm to about 250 nm. Silicone polymers having such particle size make a silicone emulsion which is stable with a wide range of components.

A convenient and useful method of preparing the silicone emulsion of the present invention is by utilizing the following procedure:

1) blending a mixture of starting silicone material selected from the group consisting of cyclic silicone oligomers such as cyclic dimehyl siloxanes known as cyclomethicone, mixed silicone hydrolyzates, silanol stopped oligomers, higher molecular weight silicone polymers, functionalized silicones and mixtures thereof with water, and anionic surfactants;

2) heating the blend obtained by mixing the starting silicone material, water and anionic surfactant to a temperature ranging from about 75 to about 98° C. for a period of time ranging from about 1 to about 5 hours;

3) cooling the anionically emulsion polymerized silicone emulsion to temperature ranging from 0 to about 25° C. for a period of time ranging from about 3 hours to about 24 hours;

4) adding a compatibilizing surfactant; and

5) adding a cationic surfactant.

The silicone polymer is comprised at a level of from about 0.01% to about 20%, more preferably from about 0.1% to about 10% of the entire composition.

Silicone Polymer

The silicone polymer of the present invention are those which provide excellent conditioning benefits to the hair. The silicone polymer is selected from the group consisting of a polyalkyl siloxane having a molecular weight of at least 20,000, a polyaryl siloxane having a molecular weight of at least 20,000, an amino-substituted siloxane having a molecular weight of at least 5,000, a silicone resin having a molecular weight of at least 5,000, and mixtures thereof.

The polyalkyl siloxanes and polyaryl siloxanes useful as silicone polymers herein include those with the following structure (I):

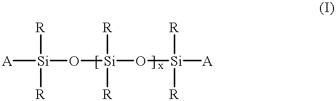

wherein R is alkyl or aryl, and x is an integer from about 200 to about 8,000 having a molecular weight of at least 20,000, more preferably at least 100,000, still more preferably at least 200,000. “A” represents groups which block the ends of the silicone chains. The alkyl or aryl groups substituted on the siloxane chain (R) or at the ends of the siloxane chains (A) can have any structure as long as the resulting silicone is dispersible, is neither irritating, toxic nor otherwise harmful when applied to the hair, is compatible with the other components of the composition, is chemically stable under normal use and storage conditions, and is capable of being deposited on and conditions the hair. Suitable A groups include hydroxy, methyl, methoxy, ethoxy, propoxy, and aryloxy. The two R groups on the silicon atom may represent the same group or different groups. Preferably, the two R groups represent the same group. Suitable R groups include methyl, ethyl, propyl, phenyl, methylphenyl and phenylmethyl. The preferred polyalkyl and polyaryl silicone polymers are polydimethylsiloxane, polydiethylsiloxane, polymethylphenylsiloxane, and derivatives thereof terminated with hydroxy and carboxyl groups. Polydimethylsiloxane, which is also known as dimethicone, and its hydroxyl terminated derivative, which is also known as dimethiconol, is especially preferred.

Also useful herein, for enhancing the shine characteristics of hair, are highly arylated silicones, such as highly phenylated polyethyl silicone having refractive indices of about 1.46 or higher, especially about 1.52 or higher. When these high refractive index silicones are used, they should be mixed with a spreading agent, such as a surfactant or a silicone resin, as described below to decrease the surface tension and enhance the film forming ability of the material.

The amino-substituted siloxanes useful as silicone polymers herein include those with the following structure (II):

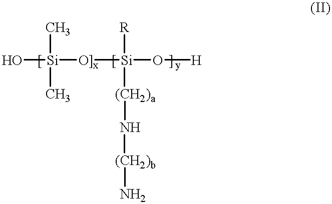

wherein R is CH₃ or OH, x and y are independent integers which depend on the desired molecular weight wherein y is not 0, a and b are independent integers from 1 to 10, and wherein the average molecular weight is at least 5,000, more preferably at least 10,000. This polymer is also known as amodimethicone.

Suitable amino-substituted siloxanes include those represented by the formula (III)

(R¹)ₐG₃₋ₐ—Si—(—OSiG₂)ₙ—(—OSiG_{b}(R¹)_{2-b)m}—O—SiG₃₋ₐ(R¹)ₐ (III)

wherein G is chosen from the group consisting of hydrogen, phenyl, OH, C₁-C₈ alkyl and preferably methyl; a denotes 0 or an integer from 1 to 3, and preferably equals 0; b denotes 0 or 1 and preferably equals 1; the sum n+m is a number from 1 to 2,000 and preferably from 50 to 150, n being able to denote a number from 0 to 1,999 and preferably from 49 to 149 and m being able to denote an integer from 1 to 2,000 and preferably from 1 to 10; R¹ is a monovalent radical of formula CqH_{2q}L in which q is an integer from 2 to 8 and L is chosen from the groups

—N(R²)CH₂—CH₂—N(R²)₂

—N(R²)₂

—N(R²)₃A⁻

—N(R²)CH₂—CH₂—NR²H₂A⁻

in which R² is chosen from the group consisting of hydrogen, phenyl, benzyl, a saturated hydrocarbon radical, preferably an alkyl radical containing from 1 to 20 carbon atoms, and A⁻ denotes a halide ion.

An especially preferred amino-substituted siloxane corresponding to formula (III) is the polymer known as “trimethylsilylamodimethicone”, of formula (IV):

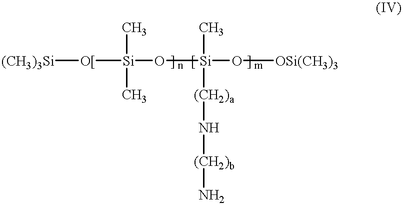

wherein n and m are independent integers of 1 or more selected depending on the desired molecular weight, a and b are independent integers from 1 to 10, and wherein the average molecular weight is at least 5,000, more preferably at least 10,000.

Other amino-substituted siloxanes which can be used are represented by the formula (V):

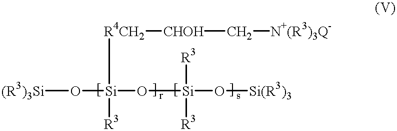

wherein R³ denotes a monovalent hydrocarbon radical having from 1 to 18 carbon atoms, preferably an alkyl or alkenyl radical such as methyl; R⁴ denotes a hydrocarbon radical, preferably a C₁-C₁₈ alkylene radical or a C₁-C₁₈, and more preferably C₁-C₈, alkyleneoxy radical; Q⁻ is a halide ion, preferably chloride; r denotes an average value from 2 to 20, preferably from 2 to 8; s denotes an average value from 20 to 200, and preferably from 20 to 50.

Also useful are silicone resins, which are highly crosslinked polymeric siloxane systems, having a molecular weight of at least 5,000, preferably at least 10,000. The cross-linking is introduced through the incorporation of trifunctional and tetrafunctional silanes with monofunctional or difunctional, or both, silanes during manufacture of the silicone resin. As is well understood in the art, the degree of crosslinking that is required in order to result in a silicone resin will vary according to the specific silane units incorporated into the silicone resin. In general, silicone materials which have a sufficient level of trifunctional and tetrafunctional siloxane monomer units, and hence, a sufficient level of crosslinking, such that they dry down to a rigid, or hard, film are considered to be silicone resins. The ratio of oxygen atoms to silicon atoms is indicative of the level of crosslinking in a particular silicone material. Silicone materials which have at least about 1.1 oxygen atoms per silicon atom will generally be silicone resins herein. Preferably, the ratio of oxygen:silicon atoms is at least about 1.2:1.0. Silanes used in the manufacture of silicone resins include monomethyl-, dimethyl-, trimethyl-, monophenyl-, diphenyl-, methylphenyl-, monovinyl-, and methylvinyl-chlorosilanes, and tetrachlorosilane, with the methyl-substituted silanes being most commonly utilized. Without being bound by theory, it is believed that the silicone resins can enhance deposition of other silicones on the hair and can enhance the glossiness of hair with high refractive index volumes.

Other useful silicone resins are silicone resin powders such as the material given the CTFA designation polymethylsilsequioxane.

Silicone resins can conveniently be identified according to a shorthand nomenclature system well known to those skilled in the art as the “MDTQ” nomenclature. Under this system, the silicone is described according to the presence of various siloxane monomer units which make up the silicone. Briefly, the symbol M denotes the monofunctional unit (CH₃)₃SiO)_{0.5}; D denotes the difunctional unit (CH₃)₂SiO; T denotes the trifunctional unit (CH₃)SiO_{1.5}; and Q denotes the quadri- or tetra-functional unit SiO₂. Primes of the unit symbols, e.g., M′, D′, T′, and Q′ denote substituents other than methyl, and must be specifically defined for each occurrence. Typical alternate substituents include groups such as vinyl, phenyl, amino, hydroxyl, etc. The molar ratios of the various units, either in terms of subscripts to the symbols indicating the total number of each type of unit in the silicone, or an average thereof, or as specifically indicated ratios in combination with molecular weight, complete the description of the silicone material under the

MDTQ system. Higher relative molar amounts of T, Q, T′ and/or Q′ to D, D′, M and/or or M′ in a silicone resin is indicative of higher levels of crosslinking. As discussed before, however, the overall level of crosslinking can also be indicated by the oxygen to silicon ratio.

The silicone resins for use herein which are preferred are MQ, MT, MTQ, MQ and MDTQ resins. Thus, the preferred silicone substituent is methyl. Especially preferred are MQ resins wherein the M:Q ratio is from about 0.5:1.0 to about 1.5:1.0.

Other silicone fluids, gums, and resins can be found in Encyclopedia of Polymer Science and Engineering, Volume 15, Second Edition, pp 204-308, John Wiley & Sons, Inc., 1989, which is incorporated herein by reference in its entirety.

Anionic Surfactant

The anionic surfactant useful for making the silicone emulsion of the present invention are those which act as an acid catalyst for polymerizing the starting silicone material, and are compatible with the remainder of components. Exemplary anionic surfactants are alkyl sulfonic acids, aryl sulfonic acids, or alkyl aryl sulfonic acids where the alkyl group ranges from one to twenty carbon atoms and the aryl group ranges from six to thirty atoms. Highly preferable anionic surfactants are those selected from the group consisting of benzene sulfonic acid, xylene sulfonic acid, dodecylbenzene sulfonic acid, and twelve to eighteen carbon atom alkyl group sulfonic acids, and mixtures thereof.

Compatibilizing Surfactant

The compatibilizing surfactant useful for making the silicone emulsion of the present invention are those which function to compatibilize the anionically emulsion polymerized silicone emulsion with the cationic surfactant. Without being bound by theory, it is believed that, if cationic surfactant is directly added to the anionic mixture obtained after the initial emulsion polymerization of starting silicone material with anionic surfactants, the anionic surfactants included in the anionically emulsion polymerized silicone emulsion having opposing ion charges to the cationic surfactants react to destroy the emulsion and/or produce undesirable precipitation. Thus, the anionically emulsion polymerized silicone emulsion obtained is treated with a compatibilizing surfactant. Useful compatibilizing surfactants are those having an HLB ratio greater than 9. Particularly useful compatibilizing surfactants are ethoxylated fatty acid esters such as polyglycerin fatty acid esters, polyoxyethylene sorbitan fatty acid esters, polyoxyethylene castor oils, polyoxyethylene secondary alkyl ethers where the alkyl group ranges from 6 to 40 carbon atoms, polyoxyethylene alkyl ethers where the alkyl group ranges from 6 to 40 carbon atoms, polyoxyethylene alkyl amines where the alkyl groups range from 6 to 40 carbon atoms and may be independently selected, polyoxyethylene alkyl amides where the alkyl groups range from 6 to 40 carbon atoms and the alkyl groups may be independently selected, amphoteric betaine surfactants, and polyoxyethylene lanolins. A particularly preferred group of surfactants are POE(4) lauryl ether, POE(9) lauryl ether, POE(23) lauryl ether, POE(20) stearyl ether, and POE(20) sorbitan mono-palmitate. Another preferred group of surfactants which may be used to compatibilize the anionic emulsion with cationic surfactants is the group consisting of lauryldimethylaminoacetic acid betaine, coco fatty amide propyldimethylaminoacetic acid betaine, 2-alkyl-N-carboxymethyl-N-hydroxyethylimidazolinium betaine, sodium N-lauroyl sarcosine, and lanolin derivatives of quaternary ammonium salts.

Cationic Surfactant

Having treated the anionically emulsion polymerized silicone emulsion with a compatibilizing surfactant, the emulsion can be treated with a cationic surfactant to obtain the cationic surfactant containing silicone emulsion of the present invention. Such silicone emulsions are compatible with a wide range of surfactants and conditioning agents of the shampoo composition of the present invention, and does not require an acyl derivative silicone suspending agent to provide a stable product. The cationic surfactants useful for making the silicone emulsion of the present invention are any known to the artisan.

Among the cationic surfactants useful herein are those corresponding to the general formula (I):

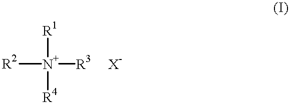

wherein R¹, R², R³, and R⁴ are independently selected from an aliphatic group of from 1 to about 22 carbon atoms or an aromatic, alkoxy, polyoxyalkylene, alkylamido, hydroxyalkyl, aryl or alkylaryl group having up to about 22 carbon atoms; and X is a salt-forming anion such as those selected from halogen, (e.g. chloride, bromide), acetate, citrate, lactate, glycolate, phosphate, nitrate, sulfonate, sulfate, alkylsulfate, and alkyl sulfonate radicals. The aliphatic groups can contain, in addition to carbon and hydrogen atoms, ether linkages, and other groups such as amino groups. The longer chain aliphatic groups, e.g., those of about 12 carbons, or higher, can be saturated or unsaturated. Preferred is when R¹, R², R³, and R⁴ are independently selected from C1 to about C22 alkyl. Nonlimiting examples of cationic surfactants useful in the present invention include the materials having the following CTFA designations: quaternium-8, quaternium-24, quaternium-26, quaternium-27, quaternium-30, quaternium-33, quaternium-43, quaternium-52, quaternium-53, quaternium-56, quaternium-60, quaternium-62, quaternium-70, quaternium-72, quaternium-75, quaternium-77, quaternium-78, quaternium-80, quaternium-81, quaternium-82, quaternium-83, quaternium-84, and mixtures thereof.

Also preferred are hydrophilically substituted cationic surfactants in which at least one of the substituents contain one or more aromatic, ether, ester, amido, or amino moieties present as substituents or as linkages in the radical chain, wherein at least one of the R¹-R⁴ radicals contain one or more hydrophilic moieties selected from alkoxy (preferably C₁-C₃ alkoxy), polyoxyalkylene (preferably C₁-C₃ polyoxyalkylene), alkylamido, hydroxyalkyl, alkylester, and combinations thereof. Preferably, the hydrophilically substituted cationic conditioning surfactant contains from 2 to about 10 nonionic hydrophile moieties located within the above stated ranges. Preferred hydrophilically substituted cationic surfactants include those of the formula (II) through (VII) below:

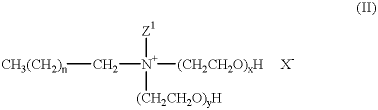

wherein n is from 8-28, x+y is from 2 to about 40, Z¹ is a short chain alkyl, preferably a C₁-C₃ alkyl, more preferably methyl, or (CH₂CH₂O)_{z}H wherein x+y+z is up to 60, and X is a salt forming anion as defined above;

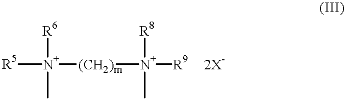

wherein m is 1 to 5, one or more of R⁵, R⁶, and R⁷ are independently an C₁-C₃₀ alkyl, the remainder are CH₂CH₂OH, one or two of R⁸, R⁹, and R¹⁰ are independently an C₁-C₃₀ alkyl, and remainder are CH₂CH₂OH, and X is a salt forming anion as mentioned above;

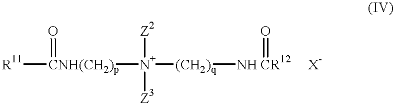

wherein Z² is an alkyl, preferably a C₁-C₃ alkyl, more preferably methyl, and Z³ is a short chain hydroxyalkyl, preferably hydroxymethyl or hydroxyethyl, p and q independently are integers from 2 to 4, inclusive, preferably from 2 to 3, inclusive, more preferably 2, R¹¹ and R¹² , independently, are substituted or unsubstituted hydrocarbyls, preferably C₁₂ -C₂₀ alkyl or alkenyl, and X is a salt forming anion as defined above;

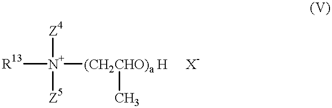

wherein R¹³ is a hydrocarbyl, preferably a C1-C3 alkyl, more preferably methyl, Z⁴ and Z⁵ are, independently, short chain hydrocarbyls, preferably C2-C4 alkyl or alkenyl, more preferably ethyl, a is from 2 to about 40, preferably from about 7 to about 30, and X is a salt forming anion as defined above;

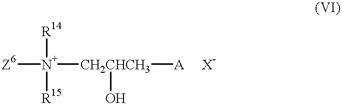

wherein R¹⁴ and R¹⁵, independently, are C₁₋₃ alkyl, preferably methyl, Z⁶ is a C₁₂ to C₂₂ hydrocarbyl, alkyl carboxy or alkylamido, and A is a protein, preferably a collagen, keratin, milk protein, silk, soy protein, wheat protein, or hydrolyzed forms thereof; and X is a salt forming anion as defined above;

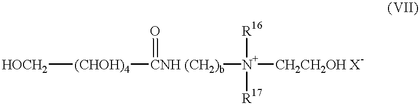

wherein b is 2 or 3, R¹⁶ and R¹⁷, independently are C₁-C₃ hydrocarbyls preferably methyl, and X is a salt forming anion as defined above. Nonlimiting examples of hydrophilically substituted cationic surfactants useful in the present invention include the materials having the following CTFA designations: quaternium-16, quaternium-61, quaternium-71, quaternium-79 hydrolyzed collagen, quaternium-79 hydrolyzed keratin, quaternium-79 hydrolyzed milk protein, quaternium-79 hydrolyzed silk, quaternium-79 hydrolyzed soy protein, and quaternium-79 hydrolyzed wheat protein. Highly preferred compounds include commercially available materials; VARIQUAT K1215 and 638 from Witco Chemical, MACKPRO KLP, MACKPRO WLW, MACKPRO MLP, MACKPRO NSP, MACKPRO NLW, MACKPRO WWP, MACKPRO NLP, MACKPRO SLP from McIntyre, ETHOQUAD 18/25, ETHOQUAD O/12PG, ETHOQUAD C/25, ETHOQUAD S/25, and ETHODUOQUAD from Akzo, DEHYQUAT SP from Henkel, and ATLAS G265 from ICI Americas.

Salts of primary, secondary, and tertiary fatty amines are also suitable cationic surfactants. The alkyl groups of such amines preferably have from about 12 to about 22 carbon atoms, and can be substituted or unsubstituted. Such amines, useful herein, include stearamido propyl dimethyl amine, diethyl amino ethyl stearamide, dimethyl stearamine, dimethyl soyamine, soyamine, myristyl amine, tridecyl amine, ethyl stearylamine, N-tallowpropane diamine, ethoxylated (with 5 moles of ethylene oxide) stearylamine, dihydroxy ethyl stearylamine, and arachidylbehenylamine. Suitable amine salts include the halogen, acetate, phosphate, nitrate, citrate, lactate, and alkyl sulfate salts. Such salts include stearylamine hydrochloride, soyamine chloride, stearylamine formate, N-tallowpropane diamine dichloride and stearamidopropyl dimethylamine citrate. Cationic amine surfactants included among those useful in the present invention are disclosed in U.S. Pat. No. 4,275,055, Nachtigal, et al., issued Jun. 23, 1981, which is incorporated by reference herein in its entirety.

The cationic surfactants for use herein may also include a plurality of ammonium quaternary moieties or amino moieties, or a mixture thereof.

Detersive Surfactants

The compositions of the present invention comprise a detersive surfactant selected from the group consisting of anionic surfactants, nonionic surfactants, amphoteric surfactants, zwitterionic surfactants, and mixtures thereof. The purpose of the detersive surfactant is to provide cleaning performance to the composition. The term detersive surfactant, as used herein, is intended to distinguish these surfactants from surfactants which are primarily emulsifying surfactants, i.e., surfactants which provide an emulsifying benefit and which have low cleansing performance. Nevertheless, however, it is recognized that many surfactants have both detersive and emulsifying properties. It is not intended to exclude emulsifying surfactants from the present invention. The detersive surfactants may or may not be the same surfactants comprised in the silicone emulsion as mentioned above.

The detersive surfactants will generally comprise from about 5% to about 50%, preferably from about 8% to about 30%, and more preferably from about 10% to about 25%, by weight of the composition.

Anionic Surfactants

Anionic surfactants useful herein include alkyl and alkyl ether sulfates. These materials have the respective formulae ROSO₃M and RO(C₂H₄O)ₓSO₃M, wherein R is alkyl or alkenyl of from about 8 to about 30 carbon atoms, x is 1 to about 10, and M is hydrogen or a cation such as ammonium, alkanolammonium (e.g., triethanolammonium), a monovalent metal cation (e.g., sodium and potassium), or a polyvalent metal cation (e.g., magnesium and calcium). Preferably, M should be chosen such that the anionic surfactant component is water soluble. The anionic surfactant should be chosen such that the Krafft temperature is about 15° C. or less, preferably about 10° C. or less, and more preferably about 0° C. or less. It is also preferred that the anionic surfactant be soluble in the composition hereof.

Krafft temperature refers to the point at which solubility of an ionic surfactant becomes determined by crystal lattice energy and heat of hydration, and corresponds to a point at which solubility undergoes a sharp, discontinuous increase with increasing temperature. Each type of surfactant will have its own characteristic Krafft temperature. Krafft temperature for ionic surfactants is, in general, well known and understood in the art. See, for example, Myers, Drew, Surfactant Science and Technology, pp. 82-85, VCH Publishers, Inc. (New York, N.Y., USA), 1988 (ISBN 0-89573-399-0), which is incorporated by reference herein in its entirety.

In the alkyl and alkyl ether sulfates described above, preferably R has from about 12 to about 18 carbon atoms in both the alkyl and alkyl ether sulfates. The alkyl ether sulfates are typically made as condensation products of ethylene oxide and monohydric alcohols having from about 8 to about 24 carbon atoms. The alcohols can be derived from fats, e.g., coconut oil, palm oil, tallow, or the like, or the alcohols can be synthetic. Lauryl alcohol and straight chain alcohols derived from coconut oil and palm oil are preferred herein. Such alcohols are reacted with 1 to about 10, and especially about 3, molar proportions of ethylene oxide and the resulting mixture of molecular species having, for example, an average of 3 moles of ethylene oxide per mole of alcohol, is sulfated and neutralized.

Specific examples of alkyl ether sulfates which can be used in the present invention are sodium and ammonium salts of coconut alkyl triethylene glycol ether sulfate; tallow alkyl triethylene glycol ether sulfate, and tallow alkyl hexaoxyethylene sulfate. Highly preferred alkyl ether sulfates are those comprising a mixture of individual compounds, said mixture having an average alkyl chain length of from about 12 to about 16 carbon atoms and an average degree of ethoxylation of from 1 to about 4 moles of ethylene oxide. Such a mixture also comprises from 0% to about 20% by weight C₁₂₋₁₃ compounds; from about 60% to about 100% by weight of C₁₄₋₁₅₋₁₆ compounds, from 0% to about 20% by weight of C₁₇₋₁₈₋₁₉ compounds; from about 3% to about 30% by weight of compounds having a degree of ethoxylation of 0; from about 45% to about 90% by weight of compounds having a degree of ethoxylation of from 1 to about 4; from about 10% to about 25% by weight of compounds having a degree of ethoxylation of from about 4 to about 8; and from about 0.1% to about 15% by weight of compounds having a degree of ethoxylation greater than about 8.

Other suitable anionic surfactants are the water-soluble salts of organic, sulfuric acid reaction products of the general formula [R¹—SO₃—M] where R¹ is selected from the group consisting of a straight or branched chain, saturated aliphatic hydrocarbon radical having from about 8 to about 24, preferably about 10 to about 18, carbon atoms; and M is as previously described above in this section. Examples of such surfactants are the salts of an organic sulfuric acid reaction product of a hydrocarbon of the methane series, including iso-, neo-, and n-paraffins, having about 8 to about 24 carbon atoms, preferably about 12 to about 18 carbon atoms and a sulfonating agent, e.g., SO₃, H₂SO₄, obtained according to known sulfonation methods, including bleaching and hydrolysis. Preferred are alkali metal and ammonium sulfonated C₁₀₋₁₈ n-paraffins.

Still other suitable anionic surfactants are the reaction products of fatty acids esterified with isethionic acid and neutralized with sodium hydroxide where, for example, the fatty acids are derived from coconut or palm oil; or sodium or potassium salts of fatty acid amides of methyl tauride in which the fatty acids, for example, are derived from coconut oil. Other similar anionic surfactants are described in U.S. Pat. No. 2,486,921, 2,486,922, and 2,396,278, which are incorporated by reference herein in their entirety.

Other anionic surfactants suitable herein are the succinates, examples of which include disodium N-octadecylsulfosuccinate; disodium lauryl sulfosuccinate; diammonium lauryl sulfosuccinate; tetra sodium N-(1,2-dicarboxyethyl)-N-octadecyl- sulfosuccinate; the diamyl ester of sodium sulfosuccinic acid; the dihexyl ester of sodium sulfosuccinic acid; and the dioctyl ester of sodium sulfosuccinic acid.

Other anionic surfactants suitable herein are those that are derived from amino acids. Nonlimiting examples of such surfactants include N-acyl-L-glutamate, N-acyl-N-methyl-alanate, N-acylsarcosinate, and their salts.

Still other useful surfactants are those that are derived from taurine, which is also known as 2-aminoethanesulfonic acid. An example of such an acid is N-acyl-N-methyl taurate.

Other suitable anionic surfactants include olefin sulfonates having about 10 to about 24 carbon atoms. The term “olefin sulfonates” is used herein to mean compounds which can be produced by the sulfonation of alpha-olefins by means of uncomplexed sulfur trioxide, followed by neutralization of the acid reaction mixture in conditions such that any sulfones which have been formed in the reaction are hydrolyzed to give the corresponding hydroxy-alkanesulfonates. The sulfur trioxide can be liquid or gaseous, and is usually, but not necessarily, diluted by inert diluents, for example by liquid SO₂, chlorinated hydrocarbons, etc., when used in the liquid form, or by air, nitrogen, gaseous SO₂, etc., when used in the gaseous form.

The alpha-olefins from which the olefin sulfonates are derived are mono-olefins having about 12 to about 24 carbon atoms, preferably about 14 to about 16 carbon atoms. Preferably, they are straight chain olefins.

In addition to the true alkene sulfonates and a proportion of hydroxy-alkanesulfonates, the olefin sulfonates can contain minor amounts of other materials, such as alkene disulfonates depending upon the reaction conditions, proportion of reactants, the nature of the starting olefins and impurities in the olefin stock and side reactions during the sulfonation process. A specific alpha-olefin sulfonate mixture of the above type is described more fully in U.S. Pat. No. 3,332,880, to Pflaumer and Kessler, issued Jul. 25, 1967, which is incorporated by reference herein in its entirety.

Another class of anionic surfactants suitable for use in the present invention are the betaalkyloxy alkane sulfonates. These compounds have the following formula:

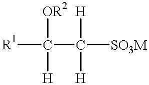

where R¹ is a straight chain alkyl group having from about 6 to about 20 carbon atoms, R² is a lower alkyl group having from about 1, preferred, to about 3 carbon atoms, and M is as hereinbefore described. Many other anionic surfactants suitable for use are described in McCutcheon's, Emulsifiers and Detergents, 1989 Annual, published by M.C. Publishing Co., and in U.S. Pat. No. 3,929,678, which descriptions are incorporated herein by reference in their entirety. Preferred anionic surfactants for use include ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, tri-ethanolamine lauryl sulfate, monoethanolamine cocoyl sulfate, monoethanolamine lauryl sulfate, sodium tridecyl benzene sulfonate, and sodium dodecyl benzene sulfonate, sodium N-lauroyl-L-glutamate, triethanolamine, N-lauryoyl-L-glutamate, sodium N-lauroyl-N-methyl taurate, sodium N-lauroyl-N-methyl--aminopropionate, and mixtures thereof.

Amphoteric and Zwitterionic Surfactants

The shampoo compositions can comprise amphoteric and/or zwitterionic surfactants.

Amphoteric surfactants for use in the shampoo compositions include the derivatives of aliphatic secondary and tertiary amines in which the aliphatic radical is straight or branched and one of the aliphatic substituents contains from about 8 to about 18 carbon atoms and one contains an anionic water solubilizing group, e.g., carboxy, sulfonate, sulfate, phosphate, or phosphonate.

Zwitterionic surfactants for use in the shampoo compositions include the derivatives of aliphatic quaternary ammonium, phosphonium, and sulfonium compounds, in which the aliphatic radicals are straight or branched, and wherein one of the aliphatic substituents contains from about 8 to about 18 carbon atoms and one contains an anionic group, e.g., carboxy, sulfonate, sulfate, phosphate, or phosphonate. A general formula for these compounds is:

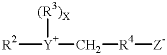

where R² contains an alkyl, alkenyl, or hydroxy alkyl radical of from about 8 to about 18 carbon atoms, from 0 to about 10 ethylene oxide moieties and from 0 to about 1 glyceryl moiety; Y is selected from the group consisting of nitrogen, phosphorus, and sulfur atoms; R³ is an alkyl or monohydroxyalkyl group containing 1 to about 3 carbon atoms; X is 1 when Y is a sulfur atom, and 2 when Y is a nitrogen or phosphorus atom; R⁴ is an alkylene or hydroxyalkylene of from 1 to about 4 carbon atoms and Z is a radical selected from the group consisting of carboxylate, sulfonate, sulfate, phos-phonate, and phosphate groups.

Examples of amphoteric and zwitterionic surfactants also include sultaines and amidosultaines. Sultaines, including amidosultaines, include for example, cocodimethylpropylsultaine, stearyldimethylpropylsultaine, lauryl-bis-(2-hydroxyethyl) propylsultaine and the like; and the amidosultaines such as cocamidodimethylpropylsultaine, stearylamidododimethylpropylsultaine, laurylamidobis-(2-hydroxyethyl) propylsultaine, and the like. Preferred are amidohydroxysultaines such as the C₁₂-C₁₈ hydrocarbyl amidopropyl hydroxysultaines, especially C₁₂-C₁₄ hydrocarbyl amido propyl hydroxysultaines, e.g., laurylamidopropyl hydroxysultaine and cocamidopropyl hydroxysultaine. Other sultaines are described in U.S. Pat. No. 3,950,417, which is incorporated herein by reference in its entirety.

Other suitable amphoteric surfactants are the aminoalkanoates of the formula R—NH(CH₂)ₙCOOM, the iminodialkanoates of the formula R—N[(CH₂)ₘCOOM]₂ and mixtures thereof; wherein n and m are numbers from 1 to about 4, R is C₈-C₂₂ alkyl or alkenyl, and M is hydrogen, alkali metal, alkaline earth metal, ammonium or alkanolammonium.

Examples of suitable aminoalkanoates include n-alkylamino-propionates and n-alkyliminodipropionates, specific examples of which include N-lauryl-beta-amino propionic acid or salts thereof, and N-lauryl-beta-imino-dipropionic acid or salts thereof, and mixtures thereof.

Other suitable amphoteric surfactants include those represented by the formula:

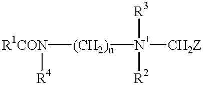

wherein R¹ is C₈-C₂₂ alkyl or alkenyl, preferably C₁₂-C₁₆, R² and R³ is independently selected from the group consisting of hydrogen, CH₂CO₂M, CH₂CH₂OH, CH₂CH₂OCH₂CH₂COOM, or (CH₂CH₂O)ₘH wherein m is an integer from 1 to about 25, and R⁴ is hydrogen, CH₂CH₂OH, or CH₂CH₂OCH₂CH₂COOM, Z is CO₂M or CH₂CO₂M, n is 2 or 3, preferably 2, M is hydrogen or a cation, such as alkali metal (e.g., lithium, sodium, potassium), alkaline earth metal (beryllium, magnesium, calcium, strontium, barium), or ammonium. This type of surfactant is sometimes classified as an imidazoline-type amphoteric surfactant, although it should be recognized that it does not necessarily have to be derived, directly or indirectly, through an imidazoline intermediate. Suitable materials of this type are marketed under the tradename MIRANOL and are understood to comprise a complex mixture of species, and can exist in protonated and non-protonated species depending upon pH with respect to species that can have a hydrogen at R². All such variations and species are meant to be encompassed by the above formula.

Examples of surfactants of the above formula are monocarboxylates and dicarboxylates. Examples of these materials include cocoamphocarboxypropionate, cocoamphocarboxypropionic acid, cocoamphocarboxyglycinate (alternately referred to as cocoamphodiacetate), and cocoamphoacetate.

Commercial amphoteric surfactants include those sold under the trade names MIRANOL C2M CONC. N.P., MIRANOL C2M CONC. O.P., MIRANOL C2M SF, MIRANOL CM SPECIAL (Miranol, Inc.); ALKATERIC 2CIB (Alkaril Chemicals); AMPHOTERGE W-2 (Lonza, Inc.); MONATERIC CDX-38, MONATERIC CSH-32 (Mona Industries); REWOTERIC AM-2C (Rewo Chemical Group); and SCHERCOTERIC MS-2 (Scher Chemicals).

Betaine surfactants, i.e. zwitterionic surfactants, suitable for use in the shampoo compositions are those represented by the formula:

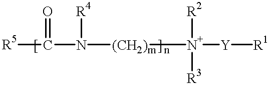

wherein:

R¹ is a member selected from the group consisting of

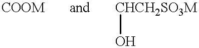

R² is lower alkyl or hydroxyalkyl; R³ is lower alkyl or hydroxyalkyl; R⁴ is a member selected from the group consisting of hydrogen and lower alkyl; R⁵ is higher alkyl or alkenyl; Y is lower alkyl, preferably methyl; m is an integer from 2 to 7, preferably from 2 to 3; n is the integer 1 or 0; M is hydrogen or a cation, as previously described, such as an alkali metal, alkaline earth metal, or ammonium. The term “lower alkyl” or “hydroxyalkyl” means straight or branch chained, saturated, aliphatic hydrocarbon radicals and substituted hydrocarbon radicals having from one to about three carbon atoms such as, for example, methyl, ethyl, propyl, isopropyl, hydroxypropyl, hydroxyethyl, and the like. The term “higher alkyl or alkenyl” means straight or branch chained saturated (i.e., “higher alkyl”) and unsaturated (i.e., “higher alkenyl”) aliphatic hydrocarbon radicals having from about eight to about 20 carbon atoms such as, for example, lauryl, cetyl, stearyl, oleyl, and the like. It should be understood that the term “higher alkyl or alkenyl” includes mixtures of radicals which may contain one or more intermediate linkages such as ether or polyether linkages or non-functional substitutents such as hydroxyl or halogen radicals wherein the radical remains of hydrophobic character.

Examples of surfactant betaines of the above formula wherein n is zero which are useful herein include the alkylbetaines such as cocodimethylcarboxymethylbetaine, lauryidimethylcarboxymethylbetaine, lauryl dimethyl-alpha-carboxyethylbetaine, cetyidimethyl-carboxmethylbetaine, lauryl-bis-(2-hydroxyethyl)carboxymethylbetaine, stearyl-bis-(2-hydroxypropyl)carboxymethylbetaine, oleyl-dimethyl-gamma-carboxypropylbetaine, lauryl-bis-(2-hydroxypropyl)-alpha-carboxyethylbetaine, etc. The sulfobetaines may be represented by cocodimethylsulfopropylbetaine, stearyidimethylsulfopropylbetaine, lauryl-bis-(2-hydroxyethyl)sulfopropylbetaine, and the like.

Specific examples of amido betaines and amidosulfo betaines useful in the shampoo compositions include the amidocarboxybetaines, such as cocamidodimethyicarboxymethylbetaine, laurylamidodi-methylcarboxymethylbetaine, cetylamidodimethylcarboxymethylbetaine, laurylamido-bis-(2-hydroxyethyl)-carboxymethylbetaine, cocamido-bis-(2-hydroxyethyl)-carboxymethylbetaine, etc. The amido sulfobetaines may be represented by cocamidodimethylsulfopropylbetaine, stearylamidodimethylsulfopropylbetaine, lauryl-amido-bis-(2-hydroxyethyl)-sulfopropylbetaine, and the like.

Nonionic Surfactants

The shampoo compostions of the present invention can comprise a nonionic surfactant. Nonionic surfactants include those compounds produced by condensation of alkylene oxide groups, hydrophilic in nature, with an organic hydrophobic compound, which may be aliphatic or alkyl aromatic in nature.

Preferred nonlimiting examples of nonionic surfactants for use in the shampoo compositions include the following:

(1) polyethylene oxide condensates of alkyl phenols, e.g., the condensation products of alkyl phenols having an alkyl group containing from about 6 to about 20 carbon atoms in either a straight chain or branched chain configuration, with ethylene oxide, the said ethylene oxide being present in amounts equal to from about 10 to about 60 moles of ethylene oxide per mole of alkyl phenol;

(2) those derived from the condensation of ethylene oxide with the product resulting from the reaction of propylene oxide and ethylene diamine products;

(3) condensation products of aliphatic alcohols having from about 8 to about 18 carbon atoms, in either straight chain or branched chain configurations, with ethylene oxide, e.g., a coconut alcohol ethylene oxide condensate having from about 10 to about 30 moles of ethylene oxide per mole of coconut alcohol, the coconut alcohol fraction having from about 10 to about 14 carbon atoms;

(4) long chain tertiary amine oxides of the formula [R¹R²R³N →O ] where R¹ contains an alkyl, alkenyl or monohydroxy alkyl radical of from about 8 to about 18 carbon atoms, from 0 to about 10 ethylene oxide moieties, and from 0 to about 1 glyceryl moiety, and R² and R³ contain from about 1 to about 3 carbon atoms and from 0 to about 1 hydroxy group, e.g., methyl, ethyl, propyl, hydroxyethyl, or hydroxypropyl radicals;

(5) long chain tertiary phosphine oxides of the formula [RR′R″P→O ] where R¹ contains an alkyl, alkenyl or monohydroxyalkyl radical ranging from about 8 to about 18 carbon atoms in chain length, from 0 to about 10 ethylene oxide moieties and from 0 to 1 glyceryl moieties and R′ and R″ are each alkyl or monohydroxyalkyl groups containing from about 1 to about 3 carbon atoms;

(6) long chain dialkyl sulfoxides containing one short chain alkyl or hydroxy alkyl radical of from 1 to about 3 carbon atoms (usually methyl) and one long hydrophobic chain which include alkyl, alkenyl, hydroxy alkyl, or keto alkyl radicals containing from about 8 to about 20 carbon atoms, from 0 to about 10 ethylene oxide moieties and from 0 to 1 glyceryl moieties;

(7) alkyl polysaccharide (APS) surfactants (e.g. alkyl polyglycosides), examples of which are described in U.S. Pat. No. 4,565,647, which is incorporated herein by reference in its entirety, and which discloses APS surfactants having a hydrophobic group with about 6 to about 30 carbon atoms and a polysaccharide (e.g., polyglycoside) as the hydrophilic group; optionally, there can be a polyalkylene-oxide group joining the hydrophobic and hydrophilic moieties; and the alkyl group (i.e., the hydrophobic moiety) can be saturated or unsaturated, branched or unbranched, and unsubstituted or substituted (e.g., with hydroxy or cyclic rings); a preferred material is alkyl polyglucoside which is commercially available from Henkel, ICI Americas, and Seppic; and

(8) polyoxyethylene alkyl ethers such as those of the formula RO(CH₂CH₂)ₙH and polyethylene glycol (PEG) glyceryl fatty esters, such as those of the formula R(O)OCH₂CH(OH)CH₂(OCH₂CH₂)ₙOH, wherein n is from 1 to about 200, preferably from about 20 to about 100, and R is an alkyl having from about 8 to about 22 carbon atoms.

Conditioning Agents

Conditioning agents known in the industry may be comprised in the present invention. Suitable conditioning agents include cationic surrfactants, such as those useful for making the silicone emulsion as described above, water soluble cationic polymers, fatty compounds, nonvolatile dispersed silicones, hydrocarbons, proteins, and mixtures thereof. These conditioning agents are comprised at a level of from about 0.01% to about 20% of the conditioning shampoo composition of the present invention.

Water Soluble Cationic Polymers

Water soluble cationic polymers are useful herein. By “water soluble” is meant a polymer which is sufficiently soluble in water to form a substantially clear solution to the naked eye at a concentration of 0.1% in water, i.e. distilled or equivalent, at 250° C. Preferably, the polymer will be sufficiently soluble to form a substantially clear solution at a 0.5% concentration, more preferably at a 1.0% concentration.

The water soluble cationic polymers hereof will generally have a weight average molecular weight which is at least about 5,000, typically at least about 10,000, and is less than about 10 million. Preferably, the molecular weight is from about 100,000 to about 2 million. The cationic polymers will generally have cationic nitrogen-containing moieties such as quaternary ammonium or cationic amino moieties, and mixtures thereof.

The cationic charge density is preferably at least about 0.1 meq/gram, more preferably at least about 0.2 meq/gram, and preferably less than about 3.0 meq/gram, more preferably less than about 2.75 meq/gram.

The cationic charge density of the cationic polymer can be determined according to the Kjeldahl Method, which is well-known to those skilled in the art. Those skilled in the art will recognize that the charge density of amino-containing polymers can vary depending upon pH and the isoelectric point of the amino groups. The charge density should be within the above limits at the pH of intended use.

Any anionic counterions can be utilized for the water soluble cationic polymers so long as the water solubility criteria is met. Suitable counterions include halides (e.g., Cl, Br, I, or F, preferably Cl, Br, or I), sulfate, and methylsulfate. Others can also be used, as this list is not exclusive.

The cationic nitrogen-containing moiety will be present generally as a substituent, on a fraction of the total monomer units of the cationic hair conditioning polymers. Thus, the water soluble cationic polymer can comprise copolymers, terpolymers, etc. of quaternary ammonium or cationic amine-substituted monomer units and other non-cationic units referred to herein as spacer monomer units. Such polymers are known in the art, and a variety can be found in International Cosmetic Ingredient Dicitonary, Fifth Edition, 1993, which is incorporated by reference herein in its entirety.

Suitable water soluble cationic polymers include, for example, copolymers of vinyl monomers having cationic amine or quaternary ammonium functionalities with water soluble spacer monomers such as acryl-amide, methacrylamide, alkyl and dialkyl acrylamides, alkyl and dialkyl methacrylamides, alkyl acrylate, alkyl methacrylate, vinyl caprolactone, and vinyl pyrrolidone. The alkyl and dialkyl substituted monomers preferably have C₁-C₇ alkyl groups, more preferably C₁-C₃ alkyl groups. Other suitable spacer monomers include vinyl esters, vinyl alcohol (made by hydrolysis of polyvinyl acetate), maleic anhydride, propylene glycol, and ethylene glycol.

The cationic amines can be primary, secondary, or tertiary amines, depending upon the particular species and the pH of the composition. In general, secondary and tertiary amines, especially tertiary amines, are preferred.

Amine-substituted vinyl monomers can be polymerized in the amine form, and then optionally can be converted to ammonium by a quaternization reaction. Amines can also be similarly quaternized subsequent to formation of the polymer. For example, tertiary amine functionalities can be quaternized by reaction with a salt of the formula R′X wherein R′ is a short chain alkyl, preferably a C₁-C₇ alkyl, more preferably a C₁-C₃ alkyl, and X is an anion which forms a water soluble salt with the quaternized ammonium.

Suitable cationic amino and quaternary ammonium monomers include, for example, vinyl compounds substituted with dialkylaminoalkyl acrylate, dialkylaminoalkyl methacrylate, monoalkylaminoalkyl acrylate, monoalkylaminoalkyl methacrylate, trialkyl methacryloxyalkyl ammonium salts, trialkyl acryloxyalkyl ammonium salts, diallyl quaternary ammonium salts, and vinyl quaternary ammonium monomers having cyclic cationic nitrogen-containing rings such as pyridinium, imidazolium, and quaternized pyrrolidone, e.g., alkyl vinyl imidazolium, alkyl vinyl pyridinium, alkyl vinyl pyrrolidone salts. The alkyl portions of these monomers are preferably lower alkyls such as the C₁-C₃ alkyls, more preferably C₁ and C₂ alkyls. Suitable amine-substituted vinyl monomers for use herein include dialkylaminoalkyl acrylate, dialkylaminoalkyl methacrylate, dialkylaminoalkyl acrylamide, and dialkylaminoalkyl methacrylamide, wherein the alkyl groups are preferably C₁-C₇ alkyl and more preferably C₁-C₃, alkyl.

The water soluble cationic polymers hereof can comprise mixtures of monomer units derived from amine-and/or quaternary ammonium-substituted monomer and/or compatible spacer monomers.

Suitable water soluble cationic polymers include, for example: copolymers of 1-vinyl-2-pyrrolidone and 1-vinyl-3-methylimidazolium salt (e.g., chloride salt), referred to in the industry by the CTFA designation as polyquaternium-16, which is commercially available from BASF Corporation under the LUVIQUAT tradename (e.g., LUVIQUAT FC 370); copolymers of 1-vinyl-2-pyrrolidone and dimethylaminoethyl methacrylate, referred to as polyquaternium-11, which is commercially available from Gaf Corporation (Wayne, N.J., USA) under the GAFQUAT tradename (e.g., GAFQUAT 755N); cationic diallyl quaternary ammonium-containing polymers, including, for example, dimethyldiallylammonium chloride homopolymer and copolymers of acrylamide and dimethyidiallylammonium chloride, referred to in the industry by the CTFA designations polyquatemium-6 and polyquaternium-7, respectively; and mineral acid salts of amino-alkyl esters of homo- and co-polymers of unsaturated carboxylic acids having from 3 to 5 carbon atoms, as described in U.S. Pat. No. 4,009,256, incorporated herein by reference.

Other water soluble cationic polymers that can be used include polysaccharide polymers, such as cationic cellulose derivatives and cationic starch derivatives. Cationic polysaccharide polymer materials suitable for use herein include those of the formula having repeating units:

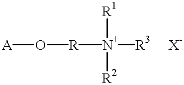

wherein A is an anhydroglucose residual group, such as a starch or cellulose anhydroglucose residual, R is an alkylene oxyalkylene, polyoxyalkylene, or hydroxyalkylene group, or combination thereof, R¹, R², and R³ independently are alkyl, aryl, alkylaryl, arylalkyl, alkoxyalkyl, or alkoxyaryl groups, each group containing up to about 18 carbon atoms, and the total number of carbon atoms for each cationic moiety (i.e., the sum of carbon atoms in R¹, R² and R³) preferably being about 20 or less, and X is an anionic counterion, e.g., halide, sulfate, nitrate, and the like.

Cationic cellulose is available from Amerchol Corp. (Edison, N.J., USA) in their Polymer JR®, LR® and SR® series of polymers, as salts of hydroxyethyl cellulose reacted with trimethyl ammonium substituted epoxide, referred to by the CTFA designation polyquaternium-10. Another type of cationic cellulose includes the polymeric quaternary ammonium salts of hydroxyethyl cellulose reacted with lauryl dimethyl ammonium-substituted epoxide, referred to by the CTFA as polyquaternium-24, and which is available from Amerchol Corp. (Edison, N.J., USA) under the tradename Polymer LM-200®.

Other water soluble cationic polymers that can be used include cationic guar gum derivatives, such as guar hydroxypropyltrimonium chloride (commercially available from Celanese Corp. in their Jaguar R series). Other materials include quaternary nitrogen-containing cellulose ethers (e.g., as described in U.S. Pat. No. 3,962,418, which is incorporated by reference herein in its entirety), and copolymers of etherified cellulose and starch (e.g., as described in U.S. Pat. No. 3,958,581, which is incorporated herein by reference in its entirety).

Preferred for use herein are water soluble cationic polymers selected from the group consisting of polyquaternium-7, polyquaternium-10, polyquaternium-11, and mixtures thereof.

Fatty Compounds

Fatty compounds including fatty alcohols, fatty acids, fatty alcohol derivatives, fatty acid derivatives, and mixtures thereof are preferred conditioning agents. It is recognized that the compounds disclosed in this section of the specification can in some instances fall into more than one classification, e.g., some fatty alcohol derivatives can also be classified as fatty acid derivatives. Also, it is recognized that some of these compounds can have properties as nonionic surfactants and can alternatively be classified as such. However, a given classification is not intendend to be a limitation on that particular compound, but is done so for convenience of classification and nomenclature. Nonlimiting examples of the fatty alcohols, fatty acids, fatty alcohol derivatives, and fatty acid derivatives are found in International Cosmetic Ingredient Dictionary, Fifth Edition, 1993, and CTFA Cosmetic Ingredient Handbook, Second Edition, 1992, both of which are incorporated by reference herein in their entirety.

The fatty alcohols useful herein are those having from about 10 to about 30 carbon atoms, preferably from about 12 to about 22 carbon atoms, and more preferably from about 16 to about 22 carbon atoms. These fatty alcohols can be straight or branched chain alcohols and can be saturated or unsaturated. Nonlimiting examples of fatty alcohols include decyl alcohol, undecyl alcohol, dodecyl, myristyl, cetyl alcohol, stearyl alcohol, isostearyl alcohol, isocetyl alcohol, behenyl alcohol, linalool, oleyl alcohol, cholesterol, cis-4-t-butylcyclohexanol, myricy alcohol and mixtures thereof. Especially preferred fatty alcohols are those selected from the group consisting of cetyl alcohol, stearyl alcohol, isostearyl alcohol, oleyl alcohol, and mixtures thereof.

The fatty acids useful herein are those having from about 10 to about 30 carbon atoms, preferably from about 12 to about 22 carbon atoms, and more preferably from about 16 to about 22 carbon atoms. These fatty acids can be straight or branched chain acids and can be saturated or unsaturated. Also included are diacids, triacids, and other multiple acids which meet the carbon number requirement herein. Also included herein are salts of these fatty acids. Nonlimiting examples of fatty acids include lauric acid, palmitic acid, stearic acid, behenic acid, arichidonic acid, oleic acid, isostearic acid, sebacic acid, and mixtures thereof. Especially preferred for use herein are the fatty acids selected from the group consisting of palmitic acid, stearic acid, and mixtures thereof.

The fatty alcohol derivatives are defined herein to include alkyl ethers of fatty alcohols, alkoxylated fatty alcohols, alkyl ethers of alkoxylated fatty alcohols, esters of fatty alcohols and mixtures thereof. Nonlimiting examples of fatty alcohol derivatives include materials such as methyl stearyl ether; 2-ethylehyl dodecyl ether; stearyl acetate; cetyl propionate; the ceteth series of compounds such as ceteth-1 through ceteth-45, which are ethylene glycol ethers of cetyl alcohol, wherein the numeric designation indicates the number of ethylene glycol moieties present; the steareth series of compounds such as steareth-1 through 100, which are ethylene glycol ethers of steareth alcohol, wherein the numeric designation indicates the number of ethylene glycol moieties present; ceteareth 1 through ceteareth-50, which are the ethylene glycol ethers of ceteareth alcohol, i.e. a mixture of fatty alcohols containing predominantly cetyl and stearyl alcohol, wherein the numeric designation indicates the number of ethylene glycol moieties present; C1-C30 alkyl ethers of the ceteth, steareth, and ceteareth compounds just described; polyoxyethylene ethers of branched alcohols such as octyldodecyl alochol, dodecylpentadecyl alcohol, hexyldecyl alcohol, and isostearyl alcohol; polyoxyethylene ethers of behenyl alcohol; PPG ethers such as PPG-9-steareth-3, PPG-11 stearyl ether, PPG-8-ceteth-1, and PPG-10 cetyl ether; and mixtures of all of the foregoing compounds. Preferred for use herein are steareth-2, steareth-4, ceteth-2, and mixtures thereof.

The fatty acid derivatives are defined herein to include fatty acid esters of the fatty alcohols as defined above in this section, fatty acid esters of the fatty alcohol derivatives as defined above in this section when such fatty alcohol derivatives have an esterifiable hydroxyl group, fatty acid esters of alcohols other than the fatty alcohols and the fatty alcohol derivatives described above in this section, hydroxy-substitued fatty acids, and mixtures thereof. Nonlimiting examples of fatty acid derivatives inlcude ricinoleic acid, glycerol monostearate, 12-hydroxy stearic acid, ethyl stearate, cetyl stearate, cetyl palmitate, polyoxyethylene cetyl ether stearate, polyoxyethylene stearyl ether stearate, polyoxyethylene lauryl ether stearate, ehtyleneglycol monostearate, polyoxyethylene monostearate, polyoxyethylene distearate, propyleneglycol monostearate, propyleneglycol distearate, trimethylolpropane distearate, sorbitan stearate, polyglyceryl stearate, dimethyl sebacate, PEG-15 cocoate, PPG-15 stearate, glyceryl monostearate, glyceryl distearate, glyceryl tristearate, PEG-8 laurate, PPG-2 isostearate, PPG-9 laurate, and mixtures thereof. Preferred for use herein are glycerol monostearate, 12-hydroxy stearic acid, and mixtures thereof.

Hydrocarbons

Hydrocarbons are useful herein as conditioning agents. Useful hydrocarbons include straight chain, cyclic, and branched chain hydrocarbons which can be either saturated or unsaturated. The hydrocarbons preferably will have from about 12 to about 40 carbon atoms, more preferably from about 12 to about 30 carbon atoms, and most preferably from about 12 to about 22 carbon atoms. Also encompassed herein are polymeric hydrocarbons of alkenyl monomers, such as polymers of C2-C6 alkenyl monomers. These polymers can be straight or branched chain polymers. The straight chain polymers will typically be relatively short in length, having a total number of carbon atoms as described above in this paragraph. The branched chain polymers can have substantially higher chain lengths. The number average molecular weight of such materials can vary widely, but will typically be up to about 500, preferably from about 200 to about 400, and more preferably from about 300 to about 350. Also useful herein are the various grades of mineral oils. Mineral oils are liquid mixtures of hydrocarbons that are obtained from petroleum. Specific examples of suitable hydrocarbon materials include paraffin oil, mineral oil, dodecane, isododecane, hexadecane, isohexadecane, eicosene, isoeicosene, tridecane, tetradecane, polybutene, polyisobutene, and mixtures thereof. Isododecane, isohexadeance, and isoeicosene are commercially available as Permethyl 99A, Permethyl 101A, and Permethyl 1082, from Presperse, South Plainfield, N.J. A copolymer of isobutene and normal butene is commercially available as Indopol H-100 from Amoco Chemicals. Preferred for use herein are hydrocarbon conditioning agents selected from the group consisting of mineral oil, isododecane, isohexadecane, polybutene, polyisobutene, and mixtures thereof.

Suspending Agents

The shampoo composition of the present invention are substantially free of acyl derivative silicone suspending agents. By the term substantially free, it is meant that the suspending agent is not included in such a sufficient amount to provide suspending effect to the silicone polymers. It is recognized that the same suspending agents can be included in smaller amounts to provide a pearlecent effect to the composition. In the present invention, it is not intended to exclude small amounts of suspending agents which could only provide a pearlecent effect, but cannot provide suspending effect to silicone polymers. Generally, suspending effect to silicone polymers cannot be seen at levels lower than about 1.5%.

The suspending agents herein include those which are present in crystalline form. These suspending agents are described in U.S. Pat. No. 4,741,855, which is incorporated herein by reference in its entirety. These preferred suspending agents include ethylene glycol esters of fatty acids preferably having from about 16 to about 22 carbon atoms such as the ethylene glycol stearates, both mono and distearate.

Optional Components

A wide variety of additional ingredients can be formulated into the present composition. These include: other conditioning agents such as hydrolysed collagen, hydrolysed keratin, proteins, plant extracts, and nutrients; hair-hold polymers; other surfactants such as anionic surfactants; thickening agents such as xanthan gum, guar gum, hydroxyethylcellulose, methylcellulose, starch and starch derivatives; viscosity modifiers such as methanolamides of long chain fatty acids such as cocomonoethanol amide; preservatives such as benzyl alcohol, methyl paraben, propyl paraben and imidazolidinyl urea; solvents such as polyvinyl alcohol, ethyl alcohol and volatile and non-volatile silicone fluids of low molecular weight; pH adjusting agents, such as citric acid, sodium citrate, succinic acid, phosphoric acid, sodium hydroxide, sodium carbonate; salts, in general, such as potassium acetate and sodium chloride; coloring agents, such as any of the FD&C or D&C dyes; hair oxidizing (bleaching) agents, such as hydrogen peroxide, perborate and persulfate salts; hair reducing agents such as the thioglycolates; perfumes; sequestering agents, such as disodium ethylenediamine tetra-acetate; and polymer plasticizing agents, such as glycerin, disobutyl adipate, butyl stearate, and propylene glycol; and ultraviolet and infrared screening and absorbing agents such as octyl salicylate. Such optional ingredients generally are used individually at levels from about 0.01% to about 10.0%, preferably from about 0.05% to about 5.0% by weight of the composition.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention. Ingredients are identified by chemical or CTFA name, or otherwise defined below.

### Examples I through V

The components shown below can be prepared by any conventional method well known in the art. A suitable method is as follows: Polyquatemium-10, when present polyethyleneglycol, mineral oil, and detersive surfactants are dispersed in water to form a homogenious mixture. To this mixture is added other ingredients except for silicone emulsion and perfume are added and agitated. The obtained mixture is passed through a heat exchanger to cool, and the silicone emulsion and perfume is added. The obtained compositions is poured into bottles to make a shampoo compositions.

COMPONENTS IN COMPOSITION
AMOUNT (%)

| EXAMPLE NO. | I | II | III | IV | V |
|---|---|---|---|---|---|
| Ammonium Laureth-3 Sulfate | 15.0 | 12.0 | 12.0 | 12.0 | 12.0 |
| Ammonium Lauryl Sulfate | 5.0 | 4.0 | 4.0 | 4.0 | 4.0 |
| Silicone Emulsion^{*1} | 6.0 | 6.0 | 6.0 | 6.0 | 6.0 |
| Polyquaternium-10 | 0.5 | 1.0 | 1.0 | 1.0 | 1.0 |
| Mineral Oil | 0.5 | 1.0 | 1.0 | 1.0 | 1.0 |
| Cetyl alcohol | 0.7 | 0.7 | 0.7 | 0.7 | 0.7 |
| Stearyl alcohol | 0.3 | 0.3 | 0.3 | 0.3 | 0.3 |
| Behenyltrimethylammonium chloride | 0 | 0 | 0 | 0.5 | 0.5 |
| Cocamidopropylbetaine | 0 | 0 | 0 | 0.5 | 0 |
| Sodioum lauroyl sarcosinate | 0 | 0 | 0 | 0 | 0.5 |
| Polyethylene glycol | 0 | 0 | 0.5 | 0.5 | 0.5 |
| Cocamide MEA | 0.9 | 0.9 | 0.7 | 0.7 | 0.7 |
| Ethyleneglycol distearate | 1.5 | 1.5 | 1.5 | 0 | 0 |
| Perfume | 0.5 | 0.5 | 0.5 | 0.5 | 0.5 |
| Preservative | 0.2 | 0.2 | 0.2 | 0.2 | 0.2 |
| Water | p.s. | p.s. | q.s. | q.s. | q.s. |
| Total | 100 | 100 | 100 | 100 | 100 |

^{*1}Silicone Emulsion: An emulsion of the following formula:
33% dimethiconol
5.4% cyclomethicone
0.8% sodium dodecylbenzene sulfonate
1.6% POE(18) nonyl phenyl ether
0.8% cetyltrimethyl ammonium chloride
0.45% preservative
57.95% water

The dimethiconol included has an average molcular weight of about 280,000 with average particle size of about 160 nm, and the level to the entire composition is 2%.

What is claimed is:

1. A shampoo composition comprising by weight:
(a) a silicone emulsion comprising:
i) from about 0.01% to about 20% of the entire composition a silicone polymer selected from the group consisting of a polyalkyl siloxane having a molecular weight of at least 20,000, a polyaryl siloxane having a molecular weight of at least 20,000, an amino-substituted siloxane having a molecular weight of at least 5,000, a silicone resin having a molecular weight of at least 5,000, and mixtures thereof;
ii) an anionic surfactant;
iii) a compatibilizing surfactant; and
iv) a cationic surfactant;
wherein the silicone polymer is dispersed as a particle having an average size of not more than about 450 nm;
(b) from about 5% to about 50% of a detersive surfactant;
(c) from about 0.1% to about 20% of a conditioning agent; and
(d) water;
wherein the composition is substantially free of acyl derivative silicone suspending agents.

2. The shampoo composition according to claim 1 wherein the silicone polymer is selected from the group consisting of a dimethiconol having a molecular weight of at least 100,000, an amodimethicone having a molecular weight of at least 10,000, and mixtures thereof.

3. The shampoo composition according to claim 1 wherein the silicone emulsion comprises the silicone polymer dispersed as a particle having an average size of from about 150 nm to about 250 nm.
